(19) United States
(12) Patent Application Publication
(10) Pub. No.: US 2025/0186824 A1
(43) Pub. Date: Jun. 12, 2025

(54) DEXTERITY SYSTEM

(71) Applicant: Neuromuscular Dynamics, LLC, La Crescenta-Montrose, CA (US)

(72) Inventor: Francisco J. Valero-Cuevas, La Crescenta, CA (US)

(73) Assignee: Neuromuscular Dynamics, LLC, La Crescenta-Montrose, CA (US)

(21) Appl. No.: 19/056,467

(22) Filed: Feb. 18, 2025

Related U.S. Application Data
(63) Continuation-in-part of application No. PCT/US2023/031079, filed on Aug. 24, 2023.
(60) Provisional application No. 63/373,421, filed on Aug. 24, 2022.

(51) Int. Cl. A63B 21/05; A63B 21/00; A63B 21/02; A63B 71/06

(52) U.S. Cl. CPC A63B 21/05; A63B 21/00061; A63B 21/023; A63B 71/0622; A63B 2071/0625; A63B 2220/24; A63B 2220/51

(57) ABSTRACT

A dexterity system includes a compressible element compressible from an extended position to a compressed position along an axis in absence of guidance in a radial direction perpendicular to the axis; a first end plate disposed at a first end of the compressible element, the first end plate enabling a user of the dexterity system to apply a compressive force to the compressible element; a second end plate disposed at a second end of the compressible element, the second end plate providing a support against the compressive force; at least one sensor configured to sense at least one parameter associated with an execution of a trial by a user operating the dexterity system; and a feedback device configured to provide the user with feedback on one or more parameters of the at least one parameter.

### CROSS-REFERENCE TO RELATED APPLICATIONS

This patent application is a U.S. national application under 35 U.S.C. § 111(a) as a continuation-in-part and claims the benefit of priority under 35 U.S.C. § 120 of PCT/US2023/031079 filed Aug. 24, 2023. PCT/US2023/031079 claims the benefit of priority under 35 U.S.C. § 119(e) to U.S. Provisional Patent Application Ser. No. 63/373,421, filed on Aug. 24, 2022. U.S. Provisional Patent Application Ser. No. 63/373,421 and PCT/US2023/031079 are incorporated by reference herein in their entirety.

### FIELD

Disclosed embodiments relate to systems and methods for developing and measuring limb force and dexterity. In particular, it relates to dexterity systems and methods that include one or more compressible elements to measure and/or develop a user's limb strength and/or dexterity. The device may be used in a regimen that allows for quantification, measurement and development of different types of motor skills.

### BACKGROUND

Adequate strength and coordination are required for individuals to perform tasks of daily living. For example, leg strength and dexterity are required when walking or standing, grasping force and grasping dexterity are required in activities such as eating, tying shoelaces and thousands of other everyday tasks. When an individual loses strength and/or dexterity, their independence and quality of life are severely compromised. Loss of leg strength and dexterity, grasping strength and dexterity, etc., can result from old age and various other health related causes such as injury or disease. The loss may be temporary, as is often the case after a person experiences an injury or orthopedic surgery and rehabilitation. The speed at which a person can regain proper operation of their limbs after surgery or disease greatly depends on the amount and quality of physical therapy that the individual receives. Personal physical therapists can be very expensive and are not available to each and every patient that requires therapy to regain strength and coordination.

In addition, there are patients that cannot afford physical therapy or do not have such services available for one reason or another, and that have to rely on self-motivation in order to develop or regain function after injury. Unfortunately, if the patient has limited access to rehabilitation services and/or facilities the opportunities to practice tasks of daily living can be limited.

There are several other reasons why an individual may wish to improve their limb strength and or dexterity. For example, an athlete may have specific goals for improving limb strength and/or dexterity; musicians that use their hands to play instruments may wish to exercise their fingers in environments where practicing their instrument is not feasible or not possible; rock climbers may wish to improve their grasping strength prior to a climb; surgeons may wish to improve their dexterity to improve their ability to perform delicate operations, etc.

Accordingly, it may be desirable to provide patients or other users with dexterity devices that they can use in different environments and that do not require them to visit a dedicated training or rehabilitation facilities.

Also, when using a dexterity device, a user may benefit from real-time or near-real time feedback. However, feedback is currently limited to simple graphical, indirect, delayed and non-intuitive types of feedback.

### SUMMARY

In general, one or more embodiments of the disclosure relate to a dexterity system, comprising: a compressible element compressible from an extended position to a compressed position along an axis in absence of guidance in a radial direction perpendicular to the axis; a first end plate disposed at a first end of the compressible element, the first end plate enabling a user of the dexterity system to apply a compressive force to the compressible element; a second end plate disposed at a second end of the compressible element, the second end plate providing a support against the compressive force; at least one sensor configured to sense at least one parameter associated with an execution of a trial by a user operating the dexterity system; and a feedback device configured to provide the user with feedback on one or more parameters of the at least one parameter.

In general, one or more embodiments of the invention relate to a a dexterity system kit, comprising: a dexterity system comprising: a compressible element compressible from an extended position to a compressed position along an axis in absence of guidance in a radial direction perpendicular to the axis, a first end plate disposed at a first end of the compressible element, the first end plate enabling a user of the dexterity system to apply a compressive force to the compressible element, a second end plate disposed at a second end of the compressible element, the second end plate providing a support against the compressive force, and a storage case for storing the dexterity system, the storage case comprising: an upper shell and a lower shell configured to: when combined, house the dexterity system, and when separated, one of the upper shell and the lower shell form a footstool for a subject using the dexterity system.

In general, one or more embodiments of the invention relate to a method for operating a dexterity system, the method comprising: sensing, by at least one sensor associated with the dexterity system, at least one parameter associated with an execution of a trial by a user operating the dexterity system; and providing, by a feedback device associated with the dexterity system, feedback on one or more parameters of the at least one parameter, wherein the dexterity system comprises: a compressible element compressible from an extended position to a compressed position along an axis in absence of guidance in a radial direction perpendicular to the axis, a first end plate disposed at a first end of the compressible element, the first end plate enabling a user of the dexterity system to apply a compressive force to the compressible element, and a second end plate disposed at a second end of the compressible element, the second end plate providing a support against the compressive force.

Other aspects of the invention will be apparent from the following description and the appended claims.

### BRIEF DESCRIPTION OF DRAWINGS

FIG. 1 schematically shows an application of a dexterity system according to one or more embodiments.

FIGS. 2A and 2B schematically show elements of a dexterity system according to one or more embodiments.

FIG. 3A, 3B, 3C, 3D, 3E1, and 3E2 provide different views of elements of a dexterity system according to one or more embodiments.

FIG. 4A shows elements of a dexterity system kit including a dexterity system and a storage case according to one or more embodiments.

FIG. 4B and 4C show elements of a storage case for a dexterity system according to one or more embodiments.

FIG. 5 shows elements of a dexterity system kit including a dexterity system and a storage case according to one or more embodiments.

FIGS. 6A-6E show flowcharts of methods according to one or more embodiments.

FIG. 7 shows a computer system, according to one or more embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third) may be used as an adjective for an element (i.e., any noun in the application). The use of ordinal numbers is not to imply or create a particular ordering of the elements nor to limit any element to being only a single element unless expressly disclosed, such as by the use of the terms “before,” “after,” “single,” and other such terminology. Rather the use of ordinal numbers is to distinguish between the elements. By way of an example, a first element is distinct from a second element, and the first element may encompass more than one element and may succeed (or precede) the second element in an ordering of elements.

In general, embodiments of the disclosure provide dexterity systems and methods for operating the dexterity systems. Embodiments of the disclosure relate to dexterity systems that, unlike previous systems, provide real-time or near real-time feedback in an intuitive, informative, and beneficial manner. Furthermore, embodiments of the disclosure relate to a dexterity system kit that combines a dexterity system with a storage case. The storage case may become a functional part of operating the dexterity system, in addition to accommodating the dexterity system for storage. A description is subsequently provided in reference to the figures.

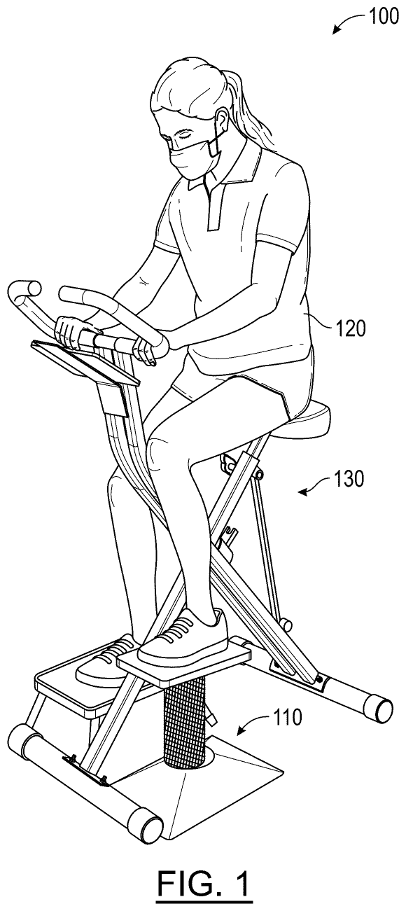

FIG. 1 schematically shows an application 100 of a dexterity system according to one or more embodiments. In the example, a dexterity system 110 for training and/or evaluation of leg strength and/or dexterity of a user 120 is shown. Other applications, while not shown, may include dexterity systems for the training and/or evaluation of other limbs including but not limited to arms, hands, fingers, etc. In the example, the user 120 sits on a support 130, allowing the user to operate the dexterity system 110 by extending her leg in a downward direction. In the example, the support 130 has the geometry of an exercise bike, but other geometries that support a stable position of the user 120 may be used without departing from the disclosure. A detailed description of the dexterity system 110 and the use of the dexterity system 110 is subsequently provided.

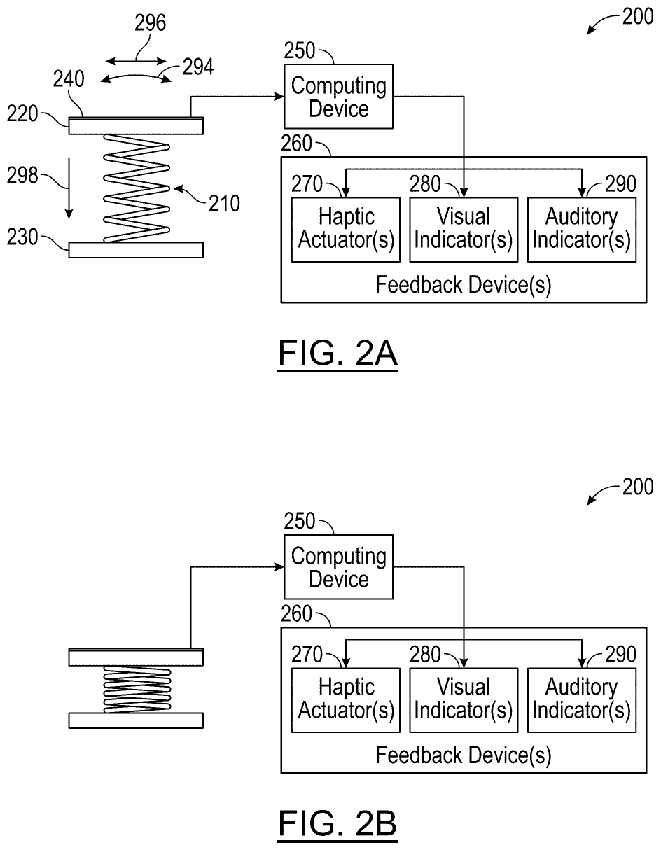

FIGS. 2A and 2B schematically show elements of a dexterity system according to one or more embodiments. The dexterity system as subsequently described applies principles of instability as a means to quantify or enhance neuromuscular control of a limb. Turning to FIG. 2A, the dexterity system 200 includes a compressible element 210, e.g., one or more springs between a first end plate 220 and a second end plate 230. In alternative embodiments the compressible element 210 may be made from a variety of pliable, flexible or resilient materials including plastic, rubber and foam rubber. Alternatively, the compressible element may be made from a combination of different compressible materials. The compressible element may return to its original extended position in the absence of an applied force.

The dexterity system 200 is designed for compression along a linear compression direction forming an axis 298, identified by an arrow, by applying an appropriate compressive force to the compressible element 210 via the first end plate 220 and/or the second end plate 230. Referring to the application 100, the foot of the user 120 may rest on the first end plate, and the user 120 may cause compression of the compressible element 210 by extending the leg in a downward direction. FIG. 2B shows the compressible element 210 in a compressed position, e.g., after the user 120 extends the leg, whereas FIG. 2A shows the compressible element in an extended position.

The user 120 may perform one or more repetitions consisting of compressing the compressible element 210, followed by a controlled release of the compressible element. The difficulty of a repetition may depend on various factors. For example, a stiff compressible element may require a higher compressive force than a compliant compressible element. Furthermore, in some embodiments, the task of compressing the compressible element 210 is complicated by an absence of guidance of the compressible element in an off-axis or radial direction (perpendicular to the linear compression direction). Accordingly, bending or flexing (buckling) in the off-axis direction may occur, unless the user applies the compressive force in a sufficiently coordinated manner. A wider and shorter compressible element 210 may be more stable in the off-axis direction than a narrower and longer compressible element 210. Accordingly, characteristics of the compressible element 210 may be tailored to a particular user's ability. Compressible elements may be interchangeable to modulate task complexity. In other words, the compressible element 210 may be tunable.

As a user improves while practicing, the execution of a repetition may become increasingly coordinated. For example, off-axis movement may be reduced, and strength may increase. In response to the improvement, the complexity of the task may be increased, for example, by tuning the characteristics of the compressible element. The compressible element may be tuned by exchanging the compressible element, adding or removing a compressible element, using active or passive means to modulate the performance of the compressible element, etc. For example, mechanical, electrical, electromechanical, electromagnetic, pneumatic, metallurgic, fluidic, auditory means, or other active means may be used to affect the properties of the compressible element 210. More specifically, tubes, rods, pistons, sleeves, fillers, bladders or other internally or externally passive means may be applied around or inside a spring-type compressible element, or pneumatic or hydraulic or otherwise fillable or pressurizable bladders inside the spring may be used to modulate off-axis stability of the compressible element 210.

In some embodiments, the dexterity system 200 includes one or more sensors 240. The one or more sensors may be used for monitoring, diagnosing, and training a person's coordination, e.g., dexterity.

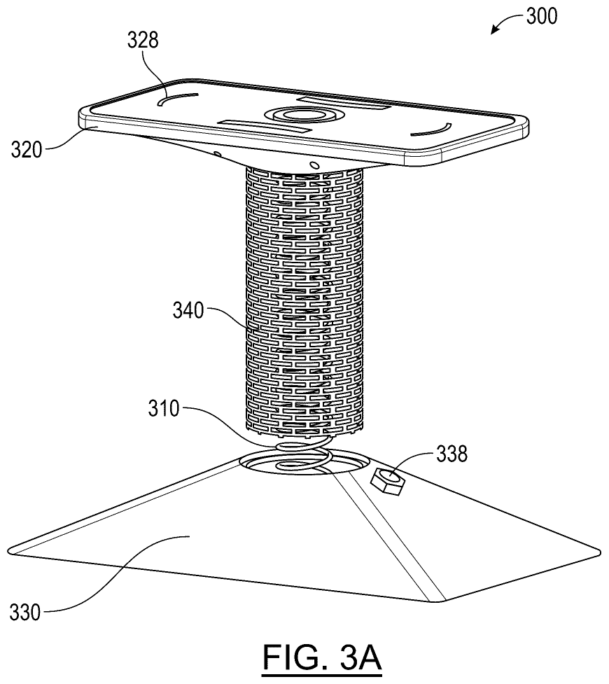

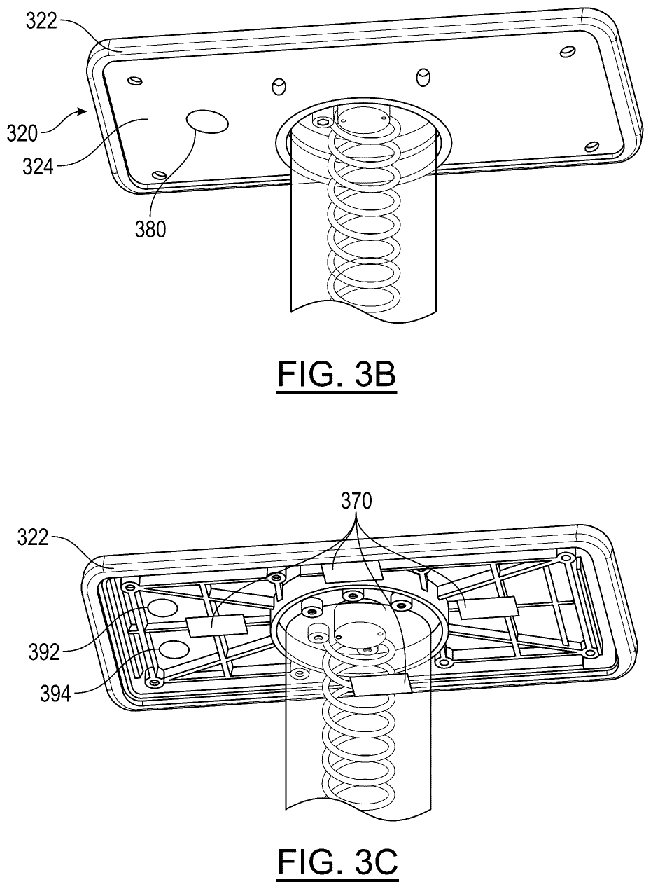

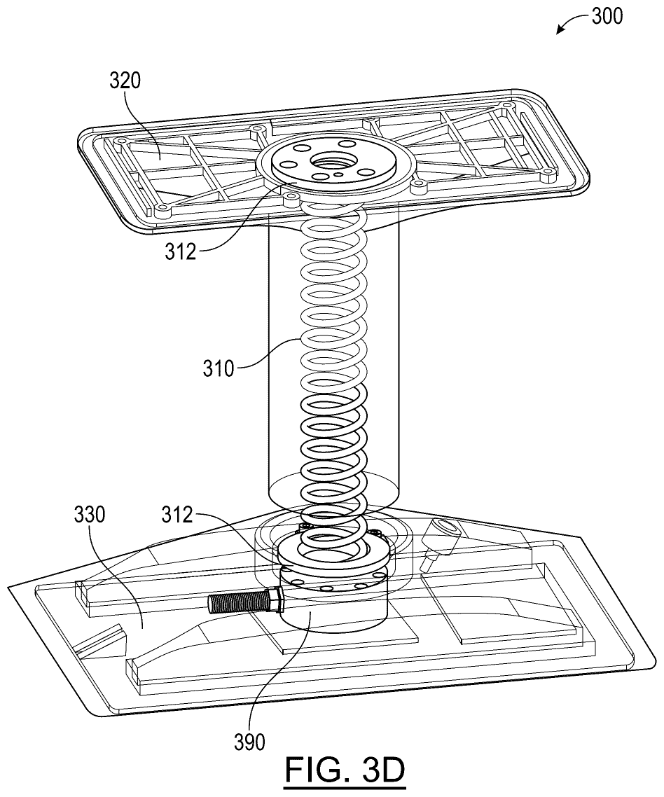

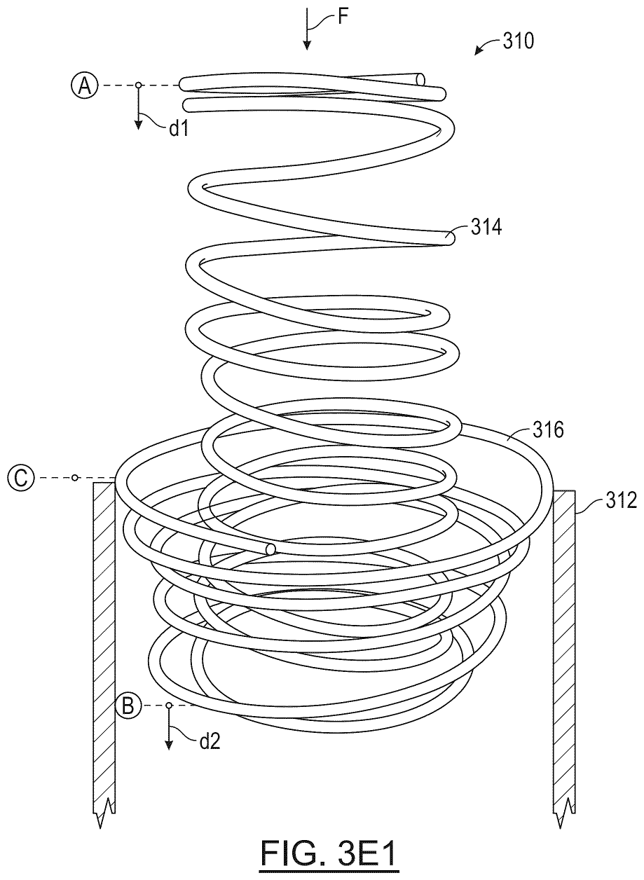

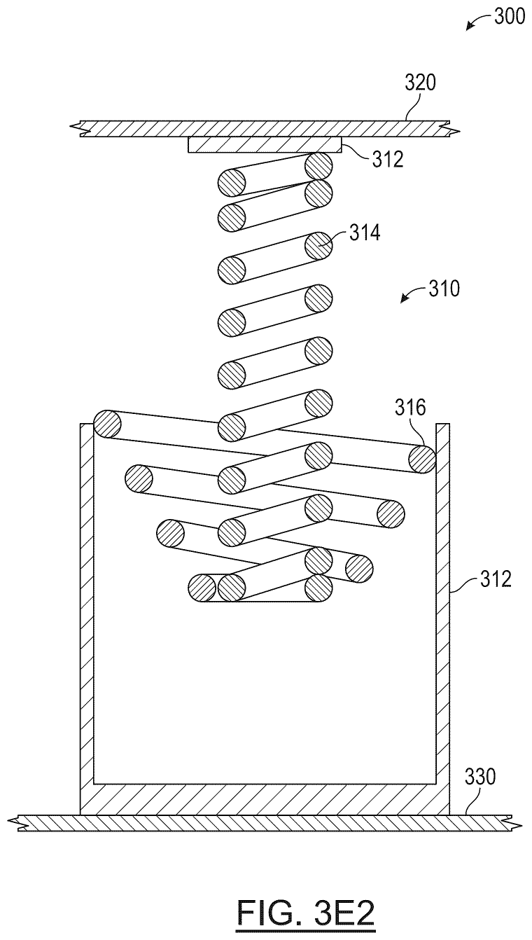

In some embodiments, the sensors 240 include one or more force sensors configured to measure the compressive force applied to the end plate(s). Any type of force sensor may be used. The force sensor may sense the compressive force, but may optionally also sense off-axis force components. A specific configuration is discussed below in reference to FIGS. 3A-3D.

In some embodiments, the sensors 240 include one or more tilt sensors configured to measure a deviation of the end plate(s) from a horizontal orientation during the compression of the compressible element 210, e.g., in two degrees of freedom. A tilt of the first end plate 220 in a first degree of freedom is identified by arrow 294, whereas a tilt in a second degree of freedom, perpendicular to the first degree of freedom, is not shown. The tilt sensor may be, for example, an inclinometer or an inertial measurement unit. Any type of tilt sensor may be used. A specific configuration is discussed below in reference to FIGS. 3A-3D.

In some embodiments, the sensors 240 include one or more lateral offset sensors configured to measure a lateral offset (in an off-axis direction, perpendicular to the linear compression direction of the axis 298) during the compression of the compressible element 210, e.g., in two degrees of freedom. The first degree of freedom of the lateral offset is identified by arrow 296, whereas the second degree of freedom (perpendicular to the first degree of freedom and perpendicular to the linear compression direction along the axis 298) is not shown. The lateral offset sensor may be, for example, an infrared, ultrasound or magnetic sensor configured to detect a translational offset of the first end plate relative to the second end plate, thereby detecting the lateral offset in the compressible element 210. The same or a similar sensor may measure movement of the first and second end plates relative to each other in the direction of the compressive force, e.g., when the compressible element is compressed during a repetition.

The dexterity system 200 may further include other sensors, without departing from the disclosure. For example, the dexterity system may include one or more sensors for user and/or location identification. Identification of the user may be beneficial to parameterize the dexterity system in a user-specific manner, as further discussed below. A QR code or barcode may be used to identify the user, and the sensor may be a camera or a dedicated QR code or barcode reader. Additionally or alternatively, biometrics (e.g., facial or fingerprint recognition) may be used to identify the user. Further, jersey or bib numbers or other markings may be detected, a transponder worn by the user, or the location of the user may also be used for identification. GPS, WIFI, cellphone or any other locally available signals or features may be used for geolocation.

The system can also be used be used near-orbit, in-orbit, around or away from the Earth for the purpose of training and treatment of space travelers, for which the corresponding space and orbital location data may be used. In addition, the physical characteristics of the location such as the amount and direction of gravity on the Moon and other planets, as well as microgravity in space, as well as induced accelerations by the trajectory and or rotation of the spacecraft (e.g., linear and angular accelerations, centrifugal and Coriolis forces, inertial forces, etc.) may be sensed and considered.

Furthermore, sensors that record physiological signals from or within the body of the user such as electromyograms, electrocardiograms, electroencephalograms, oxygen saturation levels, blood pressure, etc., may be used. Similarly, sensors for limb and joint angles and displacements, skin properties and other physiological signals indicative or informative of body biomechanical and neurological function, as well as of health, disease or recovery may be used.

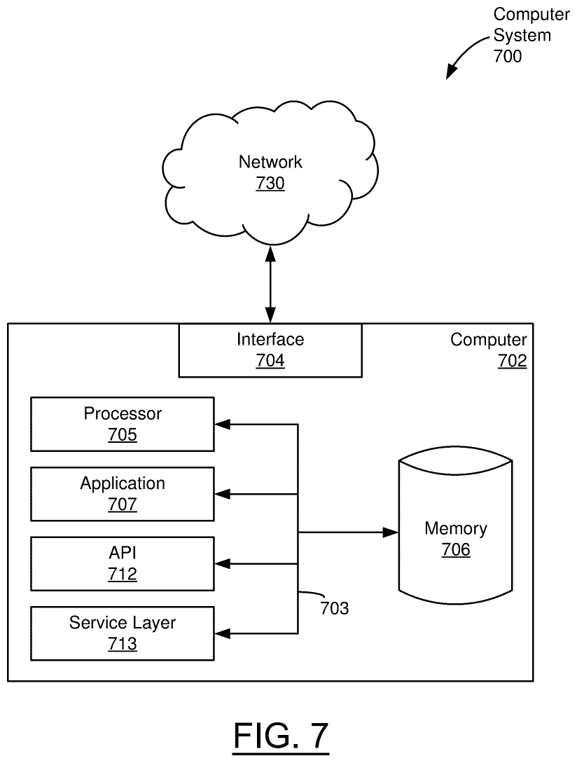

In some embodiments, the system includes a computing device 250. The computing device may be any type of computing device, e.g., an embedded system, a tablet computing device (or other type of portable computing device), etc. A description of examples of computing devices is provided below in reference to FIG. 7. The computing device may receive sensor data from the sensors 240, e.g., using wired and/or wireless interfaces. Any types of interfaces to the sensors 240 may be used. The computing device 250 may process the sensor data to determine outputs to feedback devices 260. In this configuration, the dexterity system 200 may provide real-time in addition to non-real-time feedback to the user using the dexterity system. Accordingly, the dexterity system 200 being used by the user may form a closed-loop system. The determining of outputs based on sensory data, different applications of the use of sensory data to generate feedback, and benefits of this operation (e.g., training benefits, diagnostic benefits, and therapeutic benefits) are described below in reference to the flowcharts. In some implementations that are relatively simple, the computing device 250 may rely on a hard-wired control logic.

In some embodiments, the dexterity system 200 includes one or more feedback devices 260. The feedback device may include, for example, one or more haptic actuators 270, one or more visual indicators 280, one or more auditory indicators 290, etc.

During operation of the dexterity system 200, the feedback device 260 may be used to convey various messages to the user. For example, an alert may be issued when the computing system begins or ends a data collection. An alert may also be issued for other reasons, such as relevant events, and to indicate a successfully performed trial/series of trials. For example, the alert may communicate a reward for adequate performance or a penalty for inadequate performance. A variety of reasons are discussed below in reference to the flowcharts. Generally speaking, an alert may be provided using any or any combination of the feedback devices (260).

The haptic actuator(s) 270 may include any types of haptic actuators in any combination. Piezo haptic actuators, eccentric rotating mass haptic actuators, linear resonance haptic actuators, thermal haptic actuators, solenoid haptic actuators, ultrasonic transducer haptic actuators, etc., may be used. Any number of haptic actuators may be used. The haptic actuators may be placed in the first end plate 220 and/or the second end plate 230 to enable the user to experience haptic feedback. The haptic actuators may be sized according to the application of the dexterity system 200. For example, smaller haptic actuators may be used for a dexterity system used to train grasp, whereas larger haptic actuators may be used for a dexterity system used to train leg movement. The haptic actuators may be placed to provide localized haptic feedback, e.g., to indicate a direction. A specific configuration is discussed below in reference to FIGS. 3A-3D, and a description of the use of the haptic actuators, e.g., in a closed-loop scenario, is provided in reference to the flowcharts.

The visual indicators 280 may include, for example, lights or displays. Lights may be placed in the first end plate 220 and/or the second end plate 230 to provide feedback to the user. Directional feedback may be provided by systematic placement of lights in different locations. Further, any type of display may be used. For example, a tablet computer display or a display built into the dexterity system may be used to provide instructions, status messages, graphs, directional symbols, etc. A specific configuration is discussed below in reference to FIGS. 3A-3D, and a description of the use of the visual indicators, e.g., in a closed-loop scenario, is provided in reference to the flowcharts.

The auditory indicators 290 may include, for example, speakers or buzzers. The auditory indicators may be built into the dexterity system, or they may be integrated into a tablet computer or other device.

The dexterity system 200 may further include additional elements such as a power supply, batteries, a charging circuit, a charging port, an inductive charging interface, a wired or wireless interface for the computing device 250, etc. The dexterity system 200 may further include a database that may be local or cloud-based. The database may further be distributed, with multiple databases being remotely connected, with or without synchronization. The database may be a medical database or a membership, team, or subscription database. The database may include entries such as training or rehabilitation protocols for different users. In conjunction with the sensor(s) for user and/or location identification, settings for operating the dexterity system (e.g., as described in the flowcharts) may be retrieved from the database. For example, the database may specify a clinical condition, the limb to be treated using the dexterity system, a documentation of past sessions, a configuration of the dexterity system to be used, etc.

FIG. 3A, 3B, 3C, and 3D shows elements of a dexterity system configured for leg evaluation, rehabilitation, and/or exercise, in accordance with some embodiments.

Turning to FIG. 3A, the dexterity system 300 includes elements of the dexterity system 200. For example, the dexterity system 300 includes a compression element 310, a first end plate 320, and a second end plate 330. The dexterity system 300 is configured to be operated by a leg, e.g., as illustrated in FIG. 1. Accordingly, the second end plate is formed by a base plate assembly 330, configured to be placed on a floor, and the first end plate is formed by a foot plate assembly 320 to be operated by the user's foot.

The base plate assembly 330 may be equipped with feet to ensure stable positioning on the floor. The base plate assembly may further include a switch 338 to enable and disable the dexterity system.

The foot plate assembly 320 may be equipped with a surface that ensures stable positioning of the foot. For example, the surface of the foot plate assembly 320 may be textured and or rubberized. The foot plate assembly may further display a logo, e.g., for branding. The foot plate assembly may also include alignment indicators 328 (indicating proper foot placement). In some embodiments, the surface is equipped with visual indicators such as LEDs, e.g., to indicate a status (power up, ready to pair with an external device such as a tablet computer, battery charge level, etc.), and/or to provide feedback. Feedback may be provided as discussed below in reference to the flowcharts.

In some embodiments, the dexterity system 300 also includes a compression element cover 340 that wraps around the compression element 310. The compression element cover 340 may be made of any material that can accommodate the length changes of the compression element 310 during operation of the dexterity system 300. The compression element cover 340 may be a skirt made of fabric, a plastic mesh, etc.

Additional details of the dexterity system 300 are subsequently discussed in reference to FIGS. 3B and 3C.

Turning to FIG. 3B, elements of the foot plate assembly 320 are shown in a bottom perspective view. The foot plate assembly includes a foot plate 322. The surface of the foot plate 322 may receive the user's foot. The foot plate may be surrounded by a bumper made of, for example, silicone or any other elastic material. The foot plate assembly 320 further includes a bottom foot plate cover 324. A cavity may be formed by the foot plate 322 and the bottom foot plate cover 324. The cavity may accommodate various components of the dexterity system 300. For example, one or more visual feedback indicators 380 may be included in the bottom foot plate cover 324. The visual feedback indicators 380 may be oriented to illuminate the floor or the base plate assembly 330. Multiple visual feedback indicators 380 may be used to provide directional guidance, e.g., while the user is operating the dexterity system 300. In addition or alternatively, symbols, e.g., directional symbols or instructions may be projected onto the floor or the base plate assembly 330 by the visual feedback indicator 380.

Further, as illustrated in FIG. 3C, showing a bottom view of the foot plate 322 with the bottom foot plate cover 324 removed, one or more haptic feedback actuators 370 may be disposed on a bottom surface of the foot plate 322. The haptic feedback actuators 370 may be selected to ensure that a sensation is transmitted to the foot even through the foot plate 322, shoe sole, shoes and a sock. In the example of a vibrating motor (which can be substituted by electric, pneumatic, electromechanical, a loudspeaker or other options) the pattern of stimulation (or duty cycle) and frequency content (carrier frequency) may be selected to elicit a useful percept in the user. For example, the shape of the duty cycle (on-off times and duration of on vs. off), and the carrier (constant or modulated) may be tuned. In the example illustrated in FIG. 3C, four haptic feedback actuators are shown 370, but any number of haptic feedback actuators may be used without departing from the disclosure. The presence of multiple haptic feedback actuators enables localized haptic feedback, e.g., to indicate a direction as further discussed below in reference to the flowcharts. Haptic feedback may, in additional or alternatively, be placed elsewhere, e.g., on a leg-worn sleeve on supportive armrests used by the user while operating the dexterity device, etc.

FIG. 3C further shows a tilt sensor 392 and a lateral offset sensor 394. While the tilt sensor and the lateral offset sensor are shown as being placed in a particular location, these sensors may be located elsewhere on the foot plate 322, without departing from the disclosure. Some sensor may alternatively or additional be placed in the base plate assembly 330. For example, a lateral offset sensor may include components in the foot plate assembly and components in the base plate assembly.

Turning to FIG. 3D, an assembly view of the dexterity system 300 is shown. The assembly view shows the interfacing of the compression element 310 with the foot plate assembly 320 and the base plate assembly using mounting flanges 312. The mounting flanges 312 may be of any type that is sufficiently strong to transmit compression forces while providing sufficient stability in off-axis directions. In some embodiments, the mounting flanges enable a quick release of the compression element to separate the dexterity system 300 into three pieces (the compression element, the foot plate assembly, and the base plate assembly) thus making the dexterity system collapsible, e.g., for storage and/or transportation. A bayonet-type mounting flange may be used, for example. Alternatively, the mounting flanges 312 may be equipped with hinges, allowing the dexterity system 300 to be folded into a flat configuration. The use of quick release-type mounting flanges further facilitates the exchange of compression elements, e.g., in order to reduce or increase the task difficulty when operating the dexterity system.

FIG. 3D further shows a force sensor 390, disposed on the base plate assembly 330. The mounting flange 312 of the base plate assembly 330 may be directly disposed on the force sensor. The force sensor 390 may be of any type, as previously described.

While not shown in FIG. 3D, the base plate assembly 330 may accommodate various other components of the dexterity system 300. For example, a computing device, a rechargeable battery, a power supply, a network interface, a feedback device such as auditory indicators, etc., may be disposed in the base plate assembly 330. A wire harness (not shown) may connect components in the base plate assembly 330 with components in the foot plate assembly 320, such as the haptic feedback actuators 370, the visual feedback indicators, 380, various sensors, etc.

As illustrated in reference to FIGS. 3E1 and 3E2, in some embodiments, the compressible element 310 between the mounting flanges 312 is an assembly that may include a single or multiple elements. For example, the compressible element may include a first element 314 and a second element 316. In some embodiments, the first element 314 and the second element 316 are mechanically connected in series such that a compressive force F applied to the compressible element 310 acts in a compressing direction on the first element and in a translational extending or lateral direction, or a rotational direction, on the second element. The details are subsequently described in reference to FIG. 3E1.

In FIG. 3E1, assume that the compressive force F is applied at a first end “A” of the compressible element 310, where the compressible element 310 interfaces with the first end plate 320. Further, assume that the compressible element 310 is mechanically grounded to the second end plate 330 at a second end “C” of the compressible element 310. The first and the second element interface at “B”. In the illustration of FIG. 3E, when F is applied, A translates by a distance di vertically, whereas C remains stationary. B translates by a distance d2<d1. As FIG. 3E1 illustrates, the application of F, thus, results in a compression of the first element 314 and an extension of the second element 316. Similar lateral translational or rotational interactions can arise from the application of force.

To accommodate the translation at B, the mounting flange 312 at the second end plate 330 is elongated in a vertical direction. In one example, B may translate inside a hollow mounting flange 312 when F is applied. The hollow flange may have a cylindrical shape.

Compressible elements that include multiple elements may have certain benefits over compressible elements that consist of a single element. Assume that a compressible element with certain buckling characteristics in the off-axis direction is desired. For simplicity, assume that the compressible element behaves like a slender column. Euler's critical load theory suggests that the critical load that causes buckling is governed by the aspect ratio (along other factors) of the slender column. For example, buckling may be caused by a smaller force when a column of the same cross section is longer. Accordingly, to obtain a dexterity system with certain stability characteristics (governed by when buckling occurs), a compressible element may need to have a certain length. For an increased compressive force, this may result in a longer (and potentially undesirably long) compressible element. In contrast, a compressible element that consists of multiple elements (e.g., a compressible element as shown in FIG. 3E1) has additional design parameters that can be used to obtain the same stability characteristics using a different (more compact) form factor. For example, for the same force-generating capability and the same stability characteristics, a multi-element compressible element may be shorter than a single-element compressible element. Similarly, for the same force-generating capability and the same length, a multi-element compressible element may have an increased instability (e.g., tendency to buckle).

Also, while FIG. 3E1 illustrates a compressible element that includes two concentrically arranged springs (the extension spring being the external spring, and the compression spring being the internal spring), other variations are within the scope of the disclosure. For example, the second element 316 may consist of one or more cables that may or may not be elastic. Even if the cable(s) are non-elastic, they may introduce additional instability by not directly and rigidly liking the first element to the mounting flange by allowing lateral or rotational displacement. Alternatively, the second element may be an elastic tube (e.g. a rubber tube) or any other non-rigid element. Similarly, the first element, while shown as a spring, could be any other type of compressible element. In one embodiment, one of the springs (e.g., 316) is a membrane or a structure that approximates a membrane, such as a set of radially arranged springs operating analogous to a trampoline.

For clarity, FIG. 3E2 shows the configuration of FIG. 3E1 in a cross-sectional side view that schematically shows the additional elements of the dexterity system 300 including the first end plate 320 and the second end plate 330.

4A, 4B, and 4C show elements of a dexterity system kit 400 including a dexterity system 300 and a storage case 410 according to one or more embodiments. FIG. 5 shows an alternative configuration of a storage case 510.

The storage case 410 includes an upper shell 420 and a lower shell 430. Latches 440 may be used to join the upper shell 420 and the lower shell 430. The storage case 410 enables efficient and economical housing, storage, protection, and/or transportation of the dexterity system 300. While the storage case as shown is for a leg dexterity system, similar storage cases may be used for other types of dexterity systems, without departing from the disclosure.

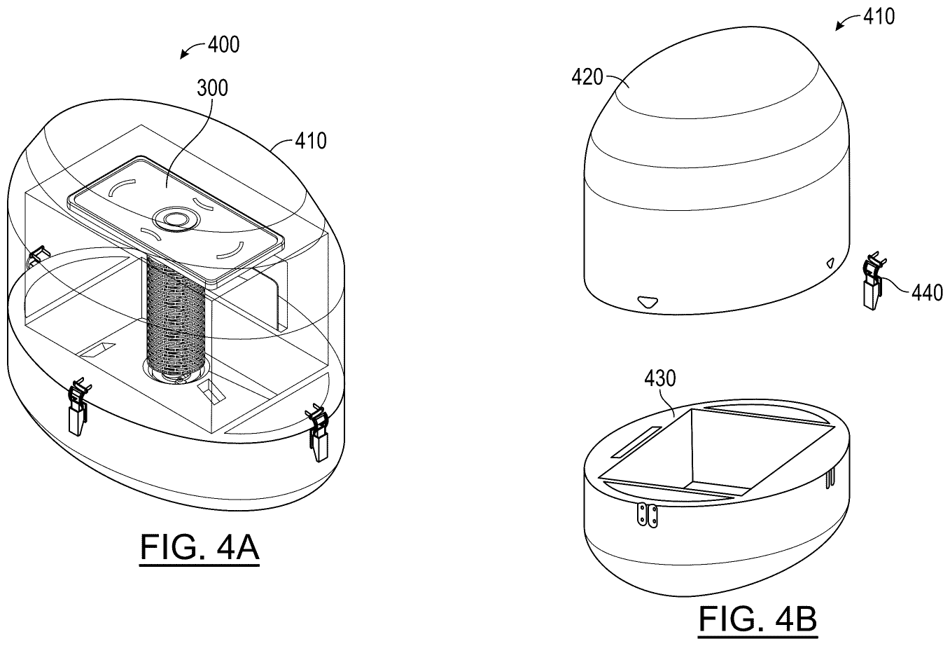

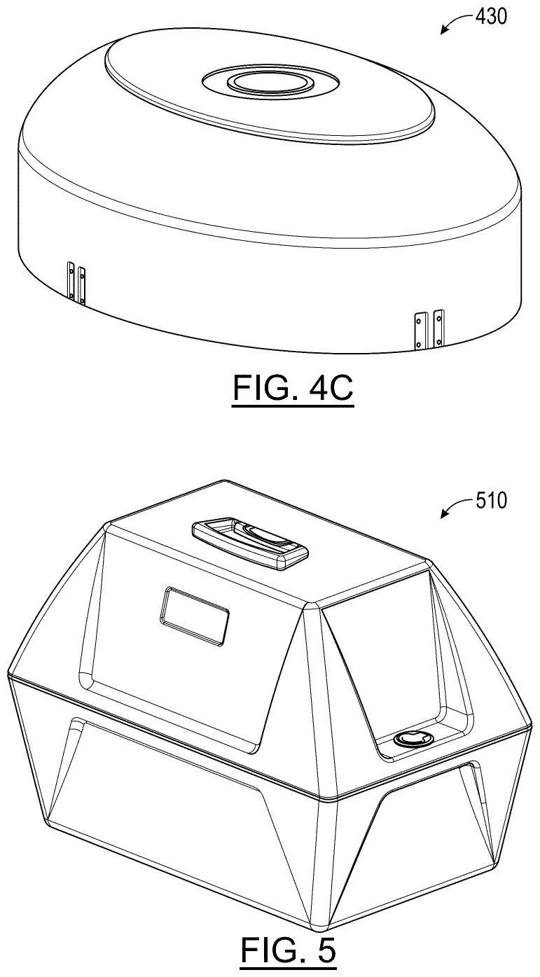

The lower shell 430 may be foam padded with cutouts or pouches to hold the dexterity system 300 and other components such as, for example, arm supports, a tablet computer or other portable computing device, chargers, documentation, etc. Furthermore, as illustrated in FIG. 4C, the lower shell 430 may be inverted to serve as a footstool. When using the dexterity system 300 with one leg, the user may stand on the footstool with the other leg. Referring to the configuration shown in FIG. 1, the user, instead of using the support 130, may stand on the footstool with the right leg, while exercising the left leg using the dexterity system.

In some embodiments, the storage case 410 includes connectors and jacks that allow re-charging of all system components (e.g., the dexterity system and a tablet computer) while enclosed or during use. The storage case 410 may be designed such that all system components, when put into the storage case 410, automatically engage connectors or connector leads for electrical re-charging and bidirectional data transfer.

In some embodiments, the storage case 410 further contains a hotspot or other communication interface to provide connectivity, e.g., to cloud enabled services.

In some embodiments, the storage case 410 contains its own power source, e.g., a battery to charge the system components such as a rechargeable battery, while the dexterity system is housed in the storage case, without the need to connect to an external power source.

While FIGS. 1, 2A, 2B, 3A-3D, 4A-4C, and 5 show various configurations of hardware components and/or software components, other configurations may be used without departing from the scope of the disclosure. For example, the dexterity device may use and exploit the capabilities of additional devices, e.g., GUI, input capabilities, connectivity and computational power of a computing device provided by the user that is itself incorporated into the “system” on a temporary basis during use.

While most aspects of the disclosure are described based on a leg dexterity device, embodiments of the disclosure also include dexterity devices for other limbs. For example, a dexterity device in accordance with embodiments of the disclosure may be designed for a finger, thumb, hand, an entire arm, an elbow, a shoulder, a hip, an ankle, a neck, etc.

Also, dexterity devices in accordance with embodiments of the disclosure may be used in zero gravity or microgravity environments such as in space, on moons and/or other planets.

Furthermore, embodiments of the disclosure may be adapted to enable use in different configurations and postures. For example, for the leg dexterity system, an adaptation may be made to eliminate the need to sit upright. Instead, the leg dexterity system may be operated with the user standing on one leg, or in a reclining chair in the lying position, from where the device may be operated much like foot pedals in a recumbent bicycle. Such a configuration also enables a configuration with a dexterity device under each foot, allowing training of both limbs without the need to switch position or move the device. It may further allow simultaneous or alternating use of the devices like “pumping” the pedals of a recumbent bicycle. Similarly, a person may use this configuration with one large device under the whole body to use the device with both feet on the same foot plate. Also, a person may be standing on two independent devices, one under each foot. The configuration may also be changed over time, for example, from a lying position to recumbent and then closer to upright sitting as the training progresses, treatment is administered, or the impairment is reduced. Alternatively, the configuration may be changed to use the same device adapted for use with legs and arms, etc.

Similarly, when traveling in aircraft or spacecraft, the device can be mounted to the bulkhead and the user be seated and secured to seats, saddles, supports and straps that firmly position the user with respect to the device even in the absence of microgravity, or when subjected to inertial forces during movement of the craft.

Embodiments of the disclosure may have one or more of the following characteristics. In one embodiment, the dexterity device is a self-contained device. The dexterity device may be operated with all visual, auditory, haptic and/or other feedback provided by the dexterity device. Accordingly, a tablet computer or smartphone may not be necessary to operate the dexterity device. Additionally, or alternatively, visual, auditory, haptic and/or otherwise feedback can be provided by the stepstool, arm rest, and other components. The feedback may be provided during an ongoing session (in real-time) or after the session. Feedback may not be provided during the session to blind the user of their performance during the use of the device, and/or to simplify use of the device. The user may also use their own smartphone or tablet computer to control the dexterity device and/or to receive information from the dexterity device.

Similarly, all sensors or other input devices may be included in any portion of the dexterity device, in the stepstool and/or in an armrest, thereby enabling autonomous operation of the dexterity device without additional devices.

In some embodiments, the dexterity device is cloud-connected, enabling a remote user to fully monitor use of the device, parameterize the device, etc. Accordingly, no additional device (e.g., a tablet computer) is needed to operate the dexterity device.

Finally, the dexterity device is easy to orient and position with respect to the limb or body part being used, under many different environmental conditions, e.g., with or without gravity.

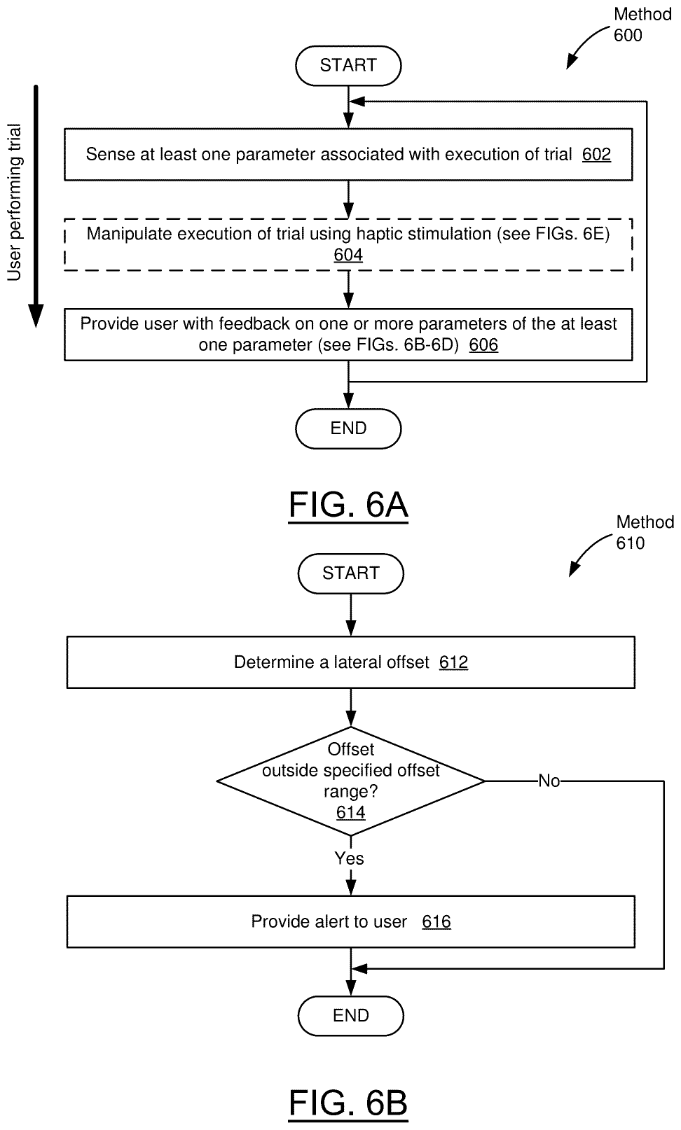

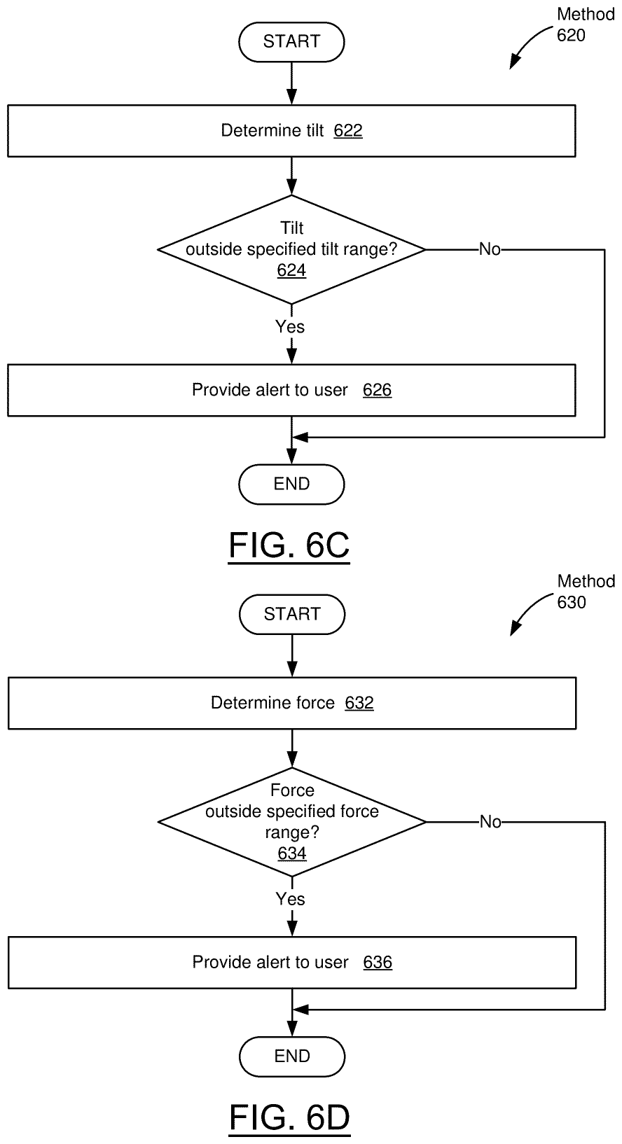

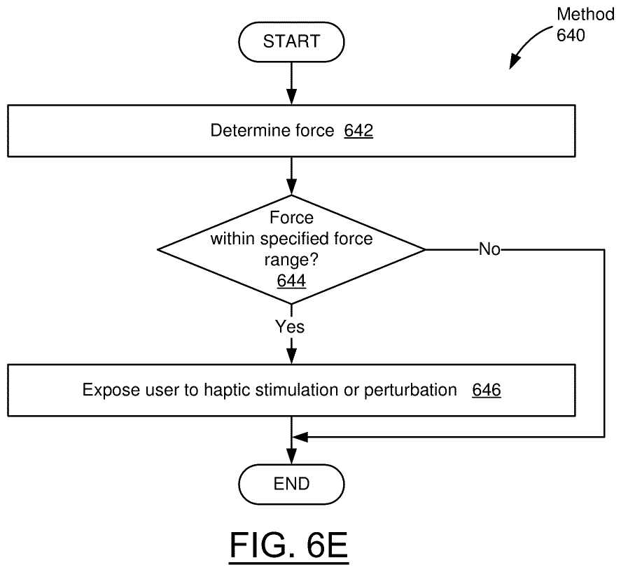

FIGS. 6A-6E show flowcharts of methods for operating a dexterity system, according to one or more embodiments. The method may be implemented using instructions stored on a non-transitory medium that may be executed by a computer system as shown in FIG. 7.

On a fundamental level, methods in accordance with embodiments of the disclosure are executed while a user performs a trial using a dexterity device. Mechanical properties of the dexterity device (e.g., of the compression element) determine the instabilities that arise, as previously discussed. Depending on the selection and tuning of a compression element, such instabilities may make the execution of the trials more challenging to a varying degree.

Execution of the subsequently discussed methods while the user is performing trials may add a closed-loop interaction to the trials. The closed loop interaction may come in the form of feedback in real-time or non-real time, and/or in the form of perturbations that may be introduced during the execution of a trial. This may make the execution of the trials more intuitive, more immediate, easier or more challenging, more productive or beneficial, and/or informative. Similarly, the device could have passive or active mobility of each part with respect to the other parts or the fixed base to displace, tilt or rotate any and all during execution either passively or motorized to make the trials more intuitive, more immediate, easier or more challenging, more productive or beneficial, and/or informative.

While the various steps in FIGS. 6A-6E are presented and described sequentially, one of ordinary skill in the art will appreciate that some or all of the steps may be executed in different orders, may be combined or omitted, and some or all of the steps may be executed in parallel. Furthermore, the steps may be performed actively or passively.

The subsequently described steps may be executed while a user of the dexterity system is performing a trial as previously described. The steps may be executed in a loop, such that they may be continuously executed for any number of trials that the user may be performing.

The described methods may be used in conjunction with any type of dexterity system, e.g., dexterity systems for legs, arms, hands/fingers, etc.

While not explicitly shown in the flowcharts, data obtained from sensors associated with the dexterity system may be recorded. Sensors associated with the dexterity system may be sensors of the dexterity system, but also other sensors, e.g., sensor worn by the user, e.g., sensors for measuring EMG, EEG, skin resistance, heart rate and/or other physiological signals.

Turning to the flowchart of method 600 shown in FIG. 6A, in Step 602, at least one parameter associated with the execution of a trial is sensed. The at least one parameter may be any type of parameter, e.g., a parameter that may be sensed by the one or more sensors of the dexterity system or sensors associated with the dexterity system. A detailed discussion of parameters that may be sensed is provided below in reference to the subsequently discussed flowcharts. Any signal obtained in Step 602 may undergo further processing such as filtering, obtaining derivatives, etc. Accordingly, any of the subsequently discussed operations that involve a sensed parameter may additionally or alternatively be performed using the processed parameter. For example, decisions may be based on not only the magnitude or range of a parameter, but also on derivatives such as velocities and accelerations, a rate of force increase or decrease, etc.

In Step 604, the execution of the trial may be manipulated, using haptic stimulation. On a fundamental level, the concept underlying the execution of Step 604 is the phenomenon of stochastic resonance. Stochastic resonance is a neurophysiological response to noise and other vibration that enhances sensibility of neurons, muscles, tendons, skin and nerves. Noise and vibration can pollute information channels in neurons muscles, tendons, skin and nerves, and make a task more difficult. Accordingly, in Step 604, such perturbations may be introduced, which may allow users to enter different domains of behavior and function, as well as alter or enhance the sensorimotor state of the user.

The manipulation performed in Step 604 may occur based on the at least one parameter sensed in Step 602, or independent from any sensed parameters. The execution of Step 604 is optional. In other words, the method 600 may also be executed without Step 604. A detailed discussion is provided below in reference to the flowcharts of FIG. 6E.

In Step 606, feedback on the one or more parameters is provided to the user. The type of feedback that may be provided is described below in reference to the flowcharts of FIGS. 6B-6D.

Turning to FIG. 6B, a method 610 for providing feedback to the user on the one or more parameters is shown. In the example, the sensing of the at least one parameter in Step 602 involves obtaining a lateral offset in, for example, one or two degrees of freedom, as previously described.

In Step 612, the lateral offset is determined, e.g., based on values obtained from sensors. An amplitude and/or a direction of the lateral offset may be determined.

In Step 614, a test is performed to determine whether the lateral offset is outside a pre-specified offset range. The pre-specified offset range may be permanently set, it may be set in a user-specific manner, it may be set based on a skill level of the user, or it may be dynamically adjusted. The offset range may be set, for example, based on benchmark values obtained from a pool of experienced users, a pool of inexperienced users, past performance of the user, performance of the user with a different limb, or in order to operate at the stability limit where it becomes challenging to operate to complete a trial. Generally speaking, it may be more challenging for the user to stay within a narrower specified offset range than in a wider specified offset range.

If the lateral offset is within the specified offset range, the execution of the method 610 may terminate without further actions.

If the lateral offset is outside the specified offset range, the method 610 may proceed with the execution of Step 616.

In Step 616, the feedback is provided to the user in the form of an alert. The user is, thus, notified when the compressible element is not sufficiently aligned (e.g., when the first end plate is not sufficiently centered above the second end plate). In some embodiments, the alert includes a location encoding that represents the direction of the lateral offset.

In one embodiment, when the first end plate is too far forward relative to the second endplate (e.g., forward relative to a foot operating the dexterity system), a stimulation pattern is activated on a front facing haptic feedback actuator of the first end plate. When the first end plate is too far backward relative to the second endplate (e.g., backward relative to a foot operating the dexterity system), a stimulation pattern is activated on a rear facing haptic feedback actuator of the first end plate. When the first end plate is too far to the left relative to the second endplate (e.g., medial relative to a right foot operating the dexterity system), a stimulation pattern is activated on a left facing haptic feedback actuator of the first end plate. When the first end plate is too far to the right relative to the second endplate (e.g., lateral relative to a right foot operating the dexterity system), a stimulation pattern is activated on a right facing haptic feedback actuator of the first end plate. The same or different types of stimulation patterns may be used for the stimulation at the different locations. Additional or alternative feedback may be provided, e.g., in the form of light signals by the visual feedback indicators described in reference to FIG. 3B.

The feedback may be provided in real-time upon the detection made in Step 614. Any type of feedback device, as previously discussed, may be activated to provide the alert.

When visual feedback is provided, the feedback may also be in non-real time. For example, there may be immediate visual feedback on the display of a tablet computer (e.g., a flashing indicator provided in real-time) but there may also be visual feedback in the form of status messages (e.g., performance quantifications, summaries, etc., which may be provided at any time, i.e., not necessarily in real-time).

Turning to FIG. 6C, a method 620 for providing feedback to the user on the one or more parameters is shown. In the example, the sensing of the at least one parameter in Step 602 involves obtaining a tilt in, for example, one or two degrees of freedom, as previously described.

In Step 622, the tilt is determined, e.g., based on values obtained from sensors. An amplitude and/or a direction of the tilt may be determined.

In Step 624, a test is performed to determine whether the tilt is outside a pre-specified tilt range. The pre-specified tilt range may be permanently set, it may be set in a user-specific manner, it may be set based on a skill level of the user, or it may be dynamically adjusted. The tilt range may be set, for example, based on benchmark values obtained from a pool of experienced users, a pool of inexperienced users, past performance of the user, performance of the user with a different limb, or in order to operate at the stability limit where it becomes challenging to operate to complete a trial. Generally speaking, it may be more challenging for the user to stay within a narrower specified tilt range than in a wider specified tilt range.

If the tilt is within the specified tilt range, the execution of the method 620 may terminate without further actions.

If the tilt is outside the specified tilt range, the method 620 may proceed with the execution of Step 626.

In Step 626, feedback is provided to the user in the form of an alert. The user is, thus, notified when the compressible element is tilted beyond the specified tilt range. In some embodiments, the alert includes a location encoding that represents the direction of the tilt.

In one embodiment, when the first end plate is tilted too far forward relative to the second endplate (e.g., downward at the front of the foot in case of a foot operating the dexterity system) a stimulation pattern is activated on a front facing haptic feedback actuator of the first end plate. When the first end plate is tilted too far backward relative to the second endplate (e.g., downward at the heel in case of a foot operating the dexterity system), a stimulation pattern is activated on a rear facing haptic feedback actuator of the first end plate. When the first end plate is tilted too far to the left relative to the second endplate (e.g., downward on the medial side of the foot, in case of a right foot operating the dexterity system), a stimulation pattern is activated on a left facing haptic feedback actuator of the first end plate. When the first end plate is too far to the right relative to the second endplate (e.g., downward on the lateral side of the foot, in case of a right foot operating the dexterity system), a stimulation pattern is activated on a right facing haptic feedback actuator of the first end plate. The same or different types of stimulation patterns may be used for the stimulation at the different locations. Additional or alternative feedback may be provided, e.g., in the form of light signals by the visual feedback indicators described in reference to FIG. 3B.

The feedback may be provided in real-time upon detection made in Step 624. Any type of feedback device, as previously discussed, may be activated to provide the alert.

When visual feedback is provided, the feedback may also be in non-real time. For example, there may be immediate visual feedback on the display of a tablet computer (e.g., a flashing indicator provided in real-time) but there may also be visual feedback in the form of status messages (e.g., performance quantifications, summaries, etc., which may be provided at any time, i.e., in not necessarily in real-time).

Turning to FIG. 6D, a method 630 for providing feedback to the user on the one or more parameters is shown. In the example, the sensing of the at least one parameter in Step 602 involves obtaining a force in, for example, the linear compression direction of the compressible element, as previously described.

In Step 632, the force is determined, e.g., based on values obtained from sensors. Alternatively, other signals that correlate with force may be used. For example, an EMG signal may be used.

In Step 634, a test is performed to determine whether the force is outside a pre-specified force range. The pre-specified force range may be permanently set, it may be set in a user-specific manner, it may be set based on a skill level of the user, or it may be dynamically adjusted. The force range may be set, for example, based on benchmark values obtained from a pool of experienced users, a pool of inexperienced users, past performance of the user, performance of the user with a different limb, or in order to operate at the stability limit where it becomes challenging to operate to complete a trial.

In some embodiments, the force range is set to encourage the user to compress the compressible element further to encourage controlling more instability. In some embodiments, the force range is set to encourage user to compress the compressible element less to encourage controlling less instability.

If the force is within the specified force range, the execution of the method 630 may terminate without further actions.

If the force is outside the specified force range, the method 630 may proceed with the execution of Step 636.

In Step 636, feedback is provided to the user in the form of an alert. The user is, thus, notified when the applied force is outside the specified force range.

In one embodiment, if the force is too low or too high in amplitude, then multiple or all feedback actuators of the first end plate may be activated using a stimulation pattern. In case of a foot operating the dexterity system the entire foot may be stimulated by the stimulation pattern. The activation may continue until the user makes corrections to reach the desired force level. The feedback may be provided in real-time upon detection made in Step 634. Any type of feedback device, as previously discussed, may be activated to provide the alert.

When visual feedback is provided, the feedback may also be in non-real time. For example, there may be immediate visual feedback on the screen of a tablet (e.g., a flashing indicator provided in real-time) but there may also be visual feedback in the form of status messages (e.g., performance quantifications, summaries, etc., which may be provided at any time, i.e., in not necessarily in real-time).

While not shown, the methods of FIGS. 6B-6D may be simultaneously executed in any combination to provide the user with feedback (real-time and/or non-real-time) on multiple variables.

Turning to FIG. 6E, a method 640 for perturbing a trial performed by the user is shown. In the example, the sensing of the at least one parameter in Step 602 involves obtaining a force in, for example, the linear compression direction of the compressible element, as previously described.

In Step 642, the force is determined, e.g., based on values obtained from sensors. Alternatively, other signals that correlate with force, such as EMG signals, may be used

In Step 644, a test is performed to determine whether the force is within a pre-specified force range. The pre-specified force range may be permanently set, it may be set in a user-specific manner, it may be set based on a skill level of the user, or it may be dynamically adjusted. The force range may be set, for example, based on benchmark values obtained from a pool of experienced users, a pool of inexperienced users, past performance of the user, performance of the user with a different limb, or in order to operate at the stability limit where it becomes challenging to operate to complete a trial. In one embodiment, the force range is set to be at the limit of instability (where the user struggles with the completion of a trial within specified boundaries, e.g., as measured based on an acceptable lateral offset and or tilt. Such boundaries may be known, based on previously performed trials, which may be statistically evaluated to set the force range, for example, based on a mean and a standard deviation.

If the force is outside the specified force range, the execution of the method 640 may terminate without further actions.

If the force is within the specified force range, the method 640 may proceed with the execution of Step 646. In some embodiments, the force may be required to be within the specified range for a certain time, e.g., a few seconds.

In Step 646, actions are taken to expose the user to a sensory perturbation. For example, multiple or all feedback actuators of the first end plate may be activated using a stimulation pattern. If the intensity of the feedback actuator is controllable, a high intensity may be used to disrupt performance of the task. In case of a foot operating the dexterity system the entire foot may be stimulated by the stimulation pattern. In other applications, further discussed below, a graded, less intense stimulation pattern may be used.

Application of the perturbation may be intended to make the use of the dexterity system more challenging to the sensorimotor system of the user. The user's response (e.g., startle or involuntary reaction) to the perturbation and recovery may be observed. Perturbations may, thus, be applied as a means to train, disrupt, rehabilitate, modify or restore sensorimotor function by modulating these sensory inputs. The perturbations and the user's reaction and/or recovery may inform the user, a coach, trainer, clinician, etc. on the levels of neuromuscular management, or disruption in response to these sensory inputs to train the user to better respond to such disruptive perturbations. The sensory perturbation may be applied at any time, e.g., mid-trial, and may involve any of the actuators associated with the dexterity system. In other words, the sensory perturbation may be applied by actuators of the dexterity system itself, or alternatively by actuators that are controlled by the dexterity system, such as electrical stimulation electrodes, e.g., on the skin surface electrodes.

An example application of the method 640 is the measurement and therapy of disability due to lower back pain. In the example application, the stimulation is used as a perturbation. The patient may be standing and holding a vertical pole in one hand, one leg on the ground and the other on the leg dexterity device performing trials, while force and standard deviation of force and other parameters such as standard deviation of accelerations and angular rates are read. As previously described, the patient may be required to operate the dexterity device at the limit of instability and hold the corresponding force for a few seconds, which then triggers the blasting of the foot plate with a stimulation pattern to disrupt the performance of the task. Measurements of the activity in the muscles of the legs, lower back, abdomen and/or neck may be performed to give feedback to the patient on training a voluntary and semi-voluntary response that reduces their lower back pain.

Another example application of the method 640 is the optimization of medication or neuromodulation (as in deep brain stimulation in Parkinson's disease). The patient may be seated, holding a vertical pole in one hand, one leg on the ground and the other on a leg dexterity device, or while using a hand dexterity device between thumb and forefinger, while force and standard deviation of force, and additional variables such as standard deviation of accelerations and angular rates are read for a given level of neuromodulation or a given dose of medication. As previously described, the patient may be required to operate the dexterity device at the limit of instability and hold the corresponding force for a few seconds, which will result in blasting the foot plate or finger platform with a stimulation pattern to disrupt the performance of the task. Measurement of the activity in the muscles of the fingers, legs, low-back and abdomen and/or neck may be performed to give feedback to the patient to train a voluntary and semi-voluntary response for that level of neuromodulation or a given dose of medication.

Yet another example application of the method 640 is the encouragement of recovery of sensory-driven leg use in patients with central or peripheral neuropathies or disruptions. The patient may be seated using a leg dexterity device, or using a hand dexterity device, while force and standard deviation of force, and other variables such as standard deviation of accelerations and angular rates are read. As previously described, the patient may be required to operate the dexterity device at the limit of instability and hold the corresponding force for a few seconds. A slow and soothing stimulation of the foot/finger platform may then be applied to promote sensory enhancement and processing. Measurement of a change in performance and repetition may result in training of the patient to better use their enhanced sensory input to promote better performance in everyday life, and when retaining the ability to walk with diabetes or Parkinson's disease.

In addition, the system can be connected to, read or act in closed loop operation with stimulators and sensors that the patient is using or has implanted. Examples include but are not limited to deep brain stimulation in the case of Parkinson's Disease or epidural and dorsal root ganglion stimulators for chronic pain or spinal cord injury or other means of neurorehabilitation, or vestibular or vagus nerve stimulation for other conditions.

While not explicitly shown in the flowcharts, the execution of any of the methods may include providing detailed feedback on the performance during one or more trials to the user and/or to another person. Such feedback on the performance may also include an assessment of the quality of the movement as performed by the user during one or more trials. At least some of these quality of movement analyses may involve significant analytics. Other, comparative quantifications may be provided in addition, including performance over time, comparison to other populations, comparison to other limbs, etc.

Embodiments of the disclosure may have one or more of the following characteristics. Embodiments of the disclosure enable quantitative methods for gauging the loss or recovery of dexterity, and methods that directly promote the recovery of dexterity. Healthy individuals, e.g., members of traveling sports teams, military units, space travelers that do not have access to physical therapy facilities and require a means to quantify and train their dexterity on their own may benefit in a similar manner. All users may benefit from the availability of feedback, which is known to motivate and promote better results as it provides an intuitive and rewarding experience that enhances the rehabilitation or training.

When a user uses the dexterity device, the device need not to be controlled remotely or in the same location by any dedicated or generic user interface device. Instead, the device's control may be driven solely by the way the user interacts with the device. The use of the dexterity device may involve an intuitive physical interaction with the device where interaction with the device is the means to control its functions.

The following is an example of the use of the device as a standalone system that does not need tablet computer or other external electronics. In the example, the user prepares to use the device and presents a barcode on their person or ID card, or transponder or marker on their person or carried by them, and/or reads a barcode on the device, stepstool, armrest or other component with their smart phone or any other transponder reading device. This identifies the subject, device, location, etc. without the need of a dedicated smartphone or tablet computer as the device, stepstool etc. The subject may use the dexterity device, including its switches, touch sensors, proximity sensors or other sensing instruments on the body of the device, stepstool, arm rest, or elsewhere without the need of a tablet computer or smartphone or other separate electronic or mechanical system. Another person (e.g., a supervisor) may or may not control the use of the device remotely or in the same location via the internet, video conferencing, etc. Data may then automatically be saved and/or uploaded to a database. A report of the usage by the user may be generated. After use, the dexterity device may enter a sleep or charging mode until the interaction with the next subject begins a new session of use.

FIG. 7 shows a computing system, according to one or more embodiments. Embodiments may be implemented on a computer system. FIG. 7 is a block diagram of a computer system 702 used to provide computational functionalities associated with described algorithms, methods, functions, processes, flows, and procedures as described in the instant disclosure, according to an implementation. The illustrated computer 702 is intended to encompass any computing device such as a high-performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 702 may include a computer that includes an input device, such as a keypad, keyboard, touch screen, or other device that can accept user information, and an output device that conveys information associated with the operation of the computer 702, including digital data, visual, or audio information (or a combination of information), or a GUI.

The computer 702 can serve in a role as a client, network component, a server, a database or other persistency, or any other component (or a combination of roles) of a computer system for performing the subject matter described in the instant disclosure. The illustrated computer 702 is communicably coupled with a network 730. In some implementations, one or more components of the computer 702 may be configured to operate within environments, including cloud-computing-based, local, global, or other environment (or a combination of environments).

At a high level, the computer 702 is an electronic computing device operable to receive, transmit, process, store, or manage data and information associated with the described subject matter. According to some implementations, the computer 702 may also include or be communicably coupled with an application server, e-mail server, web server, caching server, streaming data server, business intelligence (BI) server, or other server (or a combination of servers).

The computer 702 can receive requests over network 730 from a client application (for example, executing on another computer 702 and responding to the received requests by processing the said requests in an appropriate software application. In addition, requests may also be sent to the computer 702 from internal users (for example, from a command console or by other appropriate access method), external or third-parties, other automated applications, as well as any other appropriate entities, individuals, systems, or computers.

Each of the components of the computer 702 can communicate using a system bus 703. In some implementations, any or all of the components of the computer 702, both hardware or software (or a combination of hardware and software), may interface with each other or the interface 704 (or a combination of both) over the system bus 703 using an application programming interface (API) 712 or a service layer 713 (or a combination of the API 712 and service layer 713. The API 712 may include specifications for routines, data structures, and object classes. The API 712 may be either computer-language independent or dependent and refer to a complete interface, a single function, or even a set of APIs. The service layer 713 provides software services to the computer 702 or other components (whether or not illustrated) that are communicably coupled to the computer 702. The functionality of the computer 702 may be accessible for all service consumers using this service layer. Software services, such as those provided by the service layer 713, provide reusable, defined business functionalities through a defined interface. For example, the interface may be software written in JAVA, C++, or other suitable language providing data in extensible markup language (XML) format or other suitable format. While illustrated as an integrated component of the computer 702, alternative implementations may illustrate the API 712 or the service layer 713 as stand-alone components in relation to other components of the computer 702 or other components (whether or not illustrated) that are communicably coupled to the computer 702. Moreover, any or all parts of the API 712 or the service layer 713 may be implemented as child or sub-modules of another software module, enterprise application, or hardware module without departing from the scope of this disclosure.

The computer 702 includes an interface 704. Although illustrated as a single interface 704 in FIG. 7, two or more interfaces 704 may be used according to particular needs, desires, or particular implementations of the computer 702. The interface 704 is used by the computer 702 for communicating with other systems in a distributed environment that are connected to the network 730. Generally, the interface 704 includes logic encoded in software or hardware (or a combination of software and hardware) and operable to communicate with the network 730. More specifically, the interface 704 may include software supporting one or more communication protocols associated with communications such that the network 730 or interface's hardware is operable to communicate physical signals within and outside of the illustrated computer 702.

The computer 702 includes at least one computer processor 705. Although illustrated as a single computer processor 705 in FIG. 7, two or more processors may be used according to particular needs, desires, or particular implementations of the computer 702. Generally, the computer processor 705 executes instructions and manipulates data to perform the operations of the computer 702 and any algorithms, methods, functions, processes, flows, and procedures as described in the instant disclosure.

The computer 702 also includes a memory 706 that holds data for the computer 702 or other components (or a combination of both) that can be connected to the network 730. For example, memory 706 can be a database storing data consistent with this disclosure. Although illustrated as a single memory 706 in FIG. 7, two or more memories may be used according to particular needs, desires, or particular implementations of the computer 702 and the described functionality. While memory 706 is illustrated as an integral component of the computer 702, in alternative implementations, memory 706 can be external to the computer 702.

The application 707 is an algorithmic software engine providing functionality according to particular needs, desires, or particular implementations of the computer 702, particularly with respect to functionality described in this disclosure. For example, application 707 can serve as one or more components, modules, applications, etc. Further, although illustrated as a single application 707, the application 707 may be implemented as multiple applications 707 on the computer 702. In addition, although illustrated as integral to the computer 702, in alternative implementations, the application 707 can be external to the computer 702.

There may be any number of computers 702 associated with, or external to, a computer system containing computer 702, each computer 702 communicating over network 730. Further, the term “client,” “user,” and other appropriate terminology may be used interchangeably as appropriate without departing from the scope of this disclosure. Moreover, this disclosure contemplates that many users may use one computer 702, or that one user may use multiple computers 702.

In some embodiments, the computer 702 is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, a cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AlaaS), and/or function as a service (FaaS).

Although the disclosure has been described with respect to a limited number of embodiments, those skilled in the art, having benefit of this disclosure, will appreciate that various other embodiments may be devised without departing from the scope of the present invention. Accordingly, the scope of the invention should be limited only by the attached claims.

1. A dexterity system, comprising:
a compressible element compressible from an extended position to a compressed position along an axis in absence of guidance in a radial direction perpendicular to the axis;
a first end plate disposed at a first end of the compressible element, the first end plate enabling a user of the dexterity system to apply a compressive force to the compressible element;
a second end plate disposed at a second end of the compressible element, the second end plate providing a support against the compressive force;
at least one sensor configured to sense at least one parameter associated with an execution of a trial by a user operating the dexterity system; and
a feedback device configured to provide the user with feedback on one or more parameters of the at least one parameter.

2. The dexterity system of claim 1, wherein the compressible element comprises a plurality of elements that are mechanically in series such that during application of the compressive force, the compressive force acts in a compressing direction on a first element of the plurality of elements and in an extending direction on a second element of the plurality of elements.

3. The dexterity system of claim 2, wherein the first element is a compression spring, directly mechanically disposed on the first end plate.

4. The dexterity system of claim 2, wherein the second element is an extension spring, directly mechanically disposed on a mounting flange that protrudes from the second end plate.

5. The dexterity system of claim 2, wherein the second element comprises at least one cable, directly mechanically disposed on a mounting flange that protrudes from the second end plate.

6. The dexterity system of claim 2, wherein the second element comprises at least one flexible tube, directly mechanically disposed on a mounting flange that protrudes from the second end plate.

7. The dexterity system of claim 1, wherein the feedback comprises real-time feedback.

8. The dexterity system of claim 1, wherein
the one or more parameters comprise a tilt of the first end plate, and
the feedback comprises an alert when the tilt is outside a specified tilt range.

9. The dexterity system of claim 8, wherein the alert comprises a location encoding that represents a direction of the tilt.

10. The dexterity system of claim 1, wherein
the one or more parameters comprise a lateral offset in the compressible element in the radial direction, and
the feedback comprises an alert when the lateral offset is outside a specified offset range.

11. The dexterity system of claim 10, wherein the alert comprises a location encoding that represents a direction of the lateral offset.

12. The dexterity system of claim 1, wherein the feedback device is further configured to provide the user with an alert indicating beginning or ending of recording data from the at least one sensor.

13. The dexterity system of claim 1, wherein
the one or more parameters comprise the compressive force, and
the feedback comprises an alert when the compressive force is outside a specified compressive force range.

14. The dexterity system of claim 1, wherein the feedback device is further configured to provide one of a haptic stimulation and a haptic perturbation to the user, based on detecting that the compressive force is within a specified compressive force range.

15. The dexterity system of claim 1, wherein the feedback device is further configured to provide the user with feedback indicating a successful completion of the trial.

16. The dexterity system of claim 1, wherein the feedback device comprises at least one selected from a group consisting of a haptic actuator, a visual indicator, and an auditory indicator.

17. The dexterity system of claim 1, wherein the feedback device comprises a haptic actuator configured to activate during execution of the trial.

18. The dexterity system of claim 1, wherein the compressible element is tunable, and wherein the tunability is provided by one selected from a group consisting of:
interchangeable elements of the compressible element, and
an actuator configured to interact with the compressible element.

19. A method for operating a dexterity system, the method comprising:
sensing, by at least one sensor associated with the dexterity system, at least one parameter associated with an execution of a trial by a user operating the dexterity system; and
providing, by a feedback device associated with the dexterity system, feedback on one or more parameters of the at least one parameter,
wherein the dexterity system comprises:
a compressible element compressible from an extended position to a compressed position along an axis in absence of guidance in a radial direction perpendicular to the axis,
a first end plate disposed at a first end of the compressible element, the first end plate enabling a user of the dexterity system to apply a compressive force to the compressible element, and
a second end plate disposed at a second end of the compressible element, the second end plate providing a support against the compressive force.

20. The method of claim 19, further comprising providing one of a haptic stimulation and a haptic perturbation to the user, based on detecting that the compressive force is within a specified compressive force range.
